(19) United States
(12) Patent Application Publication
(10) Pub. No.: US 2022/0088050 A1
(43) Pub. Date: Mar. 24, 2022

(54) TREATMENT OF FATTY LIVER DISEASE

(71) Applicant: Avogadro Development Corp., New York, NY (US)

(72) Inventors: Steven B. Landau, Wellesley, MA (US); Christopher R. Shepard, New York, NY (US)

(21) Appl. No.: 17/420,808

(22) PCT Filed: Jan. 8, 2020
(86) PCT No.: PCT/US2020/012709

§ 371 Date: Jul. 6, 2021

Related U.S. Application Data
(60) Provisional application No. 62/789,874, filed on Jan. 8, 2019.

(51) Int. Cl. A61K 31/711; A61P 1/16

(52) U.S. Cl. CPC A61K 31/711; A61P 1/16

(57) ABSTRACT

The present invention relates to a method of treating fatty liver disease in a subject in need thereof comprising administering to the subject a therapeutically effective amount of a compound of Formula (I) 5′-N¹N²N³N⁴N⁵N⁶-N⁷N⁸-dC^{m}-dG^{c}-N¹¹N¹²N¹³N¹⁴N¹⁵N¹⁶-N¹⁷N¹⁸-3′. The fatty liver disease can be non-alcoholic fatty liver disease, such as simple fatty liver disease or NASH. The invention also relates to the use of adiponectin as a biomarker for identifying subjects more likely to respond to treatment with the compound on Formula (I) 5′-N¹N²N³N⁴N⁵N⁶-N⁷N⁸-dC^{m}-dG^{c}-N¹¹N¹²N¹³N¹⁴N¹⁵N¹⁶-N¹⁷N¹⁸-3′ and for assessing response of the subject during treatment with the compound of Formula (I) 5′-N¹N²N³N⁴N⁵N⁶-N⁷N⁸-dC^{m}-dG^{c}-N¹¹N¹²N¹³N¹⁴N¹⁵N¹⁶-N¹⁷N¹⁸-3′.

### RELATED APPLICATION

This application claims the benefit of U.S. Provisional Application No. 62/789,874, filed on Jan. 8, 2019.

### BACKGROUND OF THE INVENTION

Alcoholic and non alcoholic fatty liver disease (also known as hepatosteatosis) is a prevalent liver condition that occurs when lipids accumulate in liver cells. The accumulation of lipids causes cellular injury, sensitization of the liver to further injuries and damage to the hepatic microvascular circulation. The etiology of fatty liver disease is associated with excessive alcohol consumption, metabolic disorders, dietary conditions, exposure to certain chemicals and medications and complications of pregnancy (e.g., preeclampsia).

Fatty liver disease is a major health burden worldwide. The prevalence of non-alcoholic fatty liver disease (NAFLD) ranges from 15% to 37% of the population and is considered the most common liver disease worldwide. Moreover, NAFLD is also believed to affect as many as 3-10% of obese children. NAFLD can progress to a more advanced liver disease such as nonalcoholic steatohepatitis (NASH), a condition characterized by liver inflammation and damage, often accompanied by fibrosis or cirrhosis of the liver which can further lead to end stage liver disease and primary liver cancer. NASH has a prevalence of 3-10% of the general population.

Currently, there are no approved treatments for NAFLD and NASH. In general, current therapies include healthy lifestyle and non-specific metabolic modulators.

### SUMMARY OF THE INVENTION

The invention relates to a method of treating fatty liver disease. The method comprising administering to a subject in need thereof a therapeutically effect amount of the compound of Formula (I) or a pharmaceutically acceptable salt thereof.

In one embodiment, the fatty liver disease is non-alcoholic fatty liver disease (NAFLD).

In a particular embodiment, the NAFLD is simple fatty liver disease.

In another embodiment, the NAFLD is non-alcoholic steatohepatitis (NASH).

In yet another embodiment the fatty liver disease is alcoholic liver disease (ALD).

In another embodiment, the present invention relates to the use of a compound of Formula (I) or a pharmaceutically acceptable salt thereof for the manufacture of a medicament for treating fatty liver disease.

In yet another embodiment, the present invention relates to the compound of Formula (I) or a pharmaceutically acceptable salt thereof for use in treating fatty liver disease.

In yet another embodiment, the present invention relates to a method of identifying the likelihood of a fatty liver disease in a subject to be responsive to administration of a compound of Formula (I), wherein the method comprises: a) obtaining or providing a plasma sample from a subject having a fatty liver disease;
b) measuring the plasma adiponectin level in the sample; and
c) comparing the plasma adiponectin level to a control level of adiponectin; wherein if the plasma adiponectin level is less than the control level the fatty liver disease is identified as being more likely to be responsive to the administration of the compound of Formula (I).

In a particular embodiment, the control level of adiponectin is 38 μg/mL

In certain embodiments, the control level of adiponectin is 38 μg/mL or less. For example, the level of adiponectin is 38 μg/ml, 37 μg/ml, 36 μg/ml, 35 μg/ml, 34 μg/ml, 33 μg/ml, 32 μg/ml, 31 μg/ml, 30 μg/ml, 29 μg/ml, 28 μg/ml, 27 μg/ml, 26 μg/ml, 25 μg/ml, 24 μg/ml, 23 μg/ml, 22 μg/ml, 21 μg/ml, 20 μg/ml, 19 μg/ml, 18 μg/ml, 17 μg/ml, 16 μg/ml, 15 μg/ml, 14 μg/ml, 13 μg/ml, 12 μg/ml, 11 μg/ml, 10 μg/ml, 9 μg/ml, 8 μg/ml, 7 μg/ml, 6 μg/ml, 5 μg/ml, 4 μg/ml, 3 μg/ml, 2 μg/ml, 1 μg/ml, 0.5 μg/ml, 0.4 μg/ml, 0.3 μg/ml, 0.2 μg/ml or 0.1 μg/ml.

In a further embodiment, the present invention relates to a method of assessing the efficacy of a compound represented by Formula (I) in treating fatty liver disease in a subject in need thereof, the method comprising:
a) detecting in a subject plasma sample at a first point in time the level of adiponectin;
b) repeating step a) during at least one subsequent point in time after administration of the compound represented by Formula (I); and
c) comparing the levels detected in steps a) and b), wherein an increased level of adiponectin relative to at least one subsequent subject plasma sample, indicates that the compound of Formula (I) treats the fatty liver disease in the subject.

### BRIEF DESCRIPTION OF THE DRAWINGS

FIG. 1 is a graph showing the change in body weight over the treatment period of the Control Group (Group 1) and the Treated Group (Group 2).

FIG. 2 is a structural formula representing a sodium salt of Bazlitoran also referred to herein as AVO010.

### DETAILED DESCRIPTION OF THE INVENTION

A description of example embodiments of the invention follows.

While this invention has been particularly shown and described with references to example embodiments thereof, it will be understood by those skilled in the art that various changes in form and details may be made therein without departing from the scope of the invention encompassed by the appended claims.

The teachings of all patents, published applications and references cited herein are incorporated by reference in their entirety.

### Compounds for Administration

The Compounds described herein for the treatment of fatty liver disease (e.g., NASH) are 18-mer 5′-to-3′ oligonucleotides represented by structural formula (I):

5′-N¹N²N³N⁴N⁵N⁶-N⁷N⁸-dC^{m}-dG^{c}-N¹¹N¹²N¹³N¹⁴N¹⁵N¹⁶-N¹⁷N¹⁸-3′ (I)

or a pharmaceutically acceptable salt thereof.

In structural formula (I):

N¹ through N⁶ and N¹¹ through N¹⁶, each independently, is a 2′-deoxoribonucleoside;

N⁷, N⁸, N¹⁷, and N¹⁸, each independently, is a ribonucleoside, for example, a 2′-methoxy-ribonucleoside;

dC^{m} is a 5-methyl-cytosine 2′-deoxoriboside (“m⁵C-dRib,” “m⁵Cyt-dRib,” or “m⁵Cyt-dRibf”) of the following structural formula:

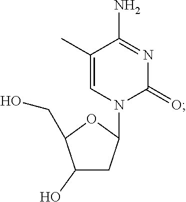 [SMILES: Cc1cn(C2CC(O)C(CO)O2)c(=O)nc1N]

dG^{c} is a 7-deaza-guanine 2′-deoxoriboside (“c⁷G-dRib,” “c⁷Gua-dRib,” or “c⁷Gua-Ribf”) of the following structural formula:

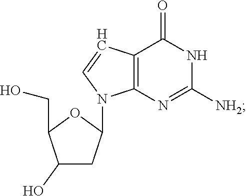 [SMILES: Nc1nc2c(ccn2C2CC(O)C(CO)O2)c(=O)[nH]1]

any two adjacent ribonucleosides are covalently linked by either a phosphodiester or a phosphorothioate linker, as represented by the following structural formulas:

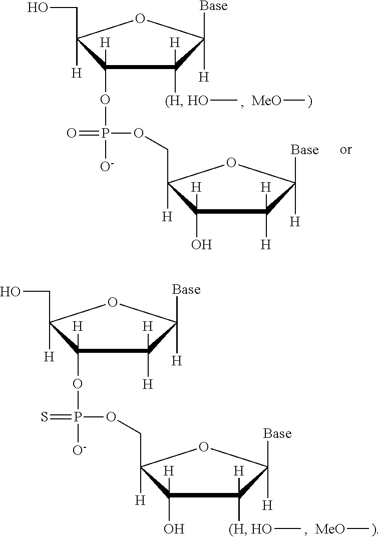

It is understood that each phosphorothioate bond introduces a chiral center designated as either “Sp” or “Rp.” Unless otherwise indicated, the compounds of the present invention that include a phosphorothioate linker can include either Sp or Rp stereoisomer. In one embodiment, all linkers in the compounds of structural formula (I) are Sp phosphorothioate linkers.

In a first example embodiment of an oligonucleotide of structural formula (I), N⁷ is a guanine 2′-methoxy-riboside (“G-Rib2Me,” “Gua-Rib2Me,” or “Gua-Ribf2Me”), represented by the following structural formula:

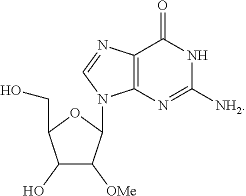 [SMILES: COC1C(O)C(CO)OC1n1cnc2c(=O)[nH]c(N)nc21]

In a second example embodiment, N⁸ is a uracil 2′-methoxy-riboside (“U-Rib2Me,” “Ura-Rib2Me,” or “Ura-Ribf2Me”) represented by the following structural formula:

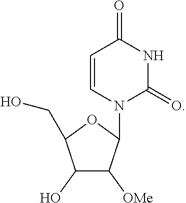 [SMILES: COC1C(O)C(CO)OC1n1ccc(=O)[nH]c1=O]

In a third example embodiment, N¹⁷ is a guanine 2′-methoxy-riboside. In a fourth example embodiment, N¹⁸ is a uracil 2′-methoxy-riboside.

In a fifth example embodiment of an oligonucleotide of structural formula (I), N⁷ is a guanine 2′-methoxy-riboside, N⁸ is a uracil 2′-methoxy-riboside, N¹⁷ is a guanine 2′-methoxy-riboside, and N¹⁸ is a uracil 2′-methoxy-riboside.

In a sixth example embodiment of an oligonucleotide of structural formula (I), N⁷ is cytosine 2′-deoxoriboside (“dC” or “dCyd”), N² is thymine 2′-deoxoriboside (“dT” or “dThd”), N³ is adenine 2′-deoxoriboside (“dA” or “dAdo”), N⁴ is thymine 2′-deoxoriboside, N⁵ is cytosine 2′-deoxoriboside, and N⁶ is thymine 2′-deoxoriboside. The values and example values of N⁷, N⁸, N¹⁷, and N¹⁸ are as described above with respect to the first through fifth example embodiments of an oligonucleotide of structural formula (I).

In a seventh example embodiment of an oligonucleotide of structural formula (I), N¹¹ is thymine 2′-deoxoriboside, N¹² is thymine 2′-deoxoriboside, N¹³ is cytosine 2′-deoxoriboside, N¹⁴ is thymine 2′-deoxoriboside, N¹⁵ is cytosine 2′-deoxoriboside, and N¹⁶ is thymine 2′-deoxoriboside. The values and example values of N¹ through N⁸, and N¹⁷ and N¹⁸ are as described above with respect to the first through sixth example embodiments of an oligonucleotide of structural formula (I).

In an eighth example embodiment of an oligonucleotide of structural formula (I), the oligonucleotide is represented by the following structural formula:

(II)
5′-dC-dT-dA-dT-dC-dT-(G-Rib2Me)-(U-Rib2Me)-dC^{m}-
dG^{c}-dT-dT-dC-dT-dC-dT-(G-Rib2Me)-(U-Rib2Me)-3′.

In a ninth example embodiment of an oligonucleotide of structural formula (I), the oligonucleotide is represented by the following structural formula:

(III)
5′-dC-Sp-dT-Sp-dA-Sp-dT-Sp-dC-Sp-dT-Sp-(G-Rib2Me)-
Sp-(U-Rib2Me)-Sp-dC^{m}-Sp-dG^{c}-Sp-dT-Sp-dT-Sp-dC-Sp-
dT-Sp-dC-Sp-dT-Sp-(G-Rib2Me)-Sp-(U-Rib2Me)-3′

or a pharmaceutically acceptable salt thereof. In a specific aspect, the oligonucleotide represented by structural formula (III) in is in the form of a sodium salt represented by structural formula (IIIA):

(IIIA)
5′-dC-Sp-dT-Sp-dA-Sp-dT-Sp-dC-Sp-dT-Sp-(G-Rib2Me)-
Sp-(U-Rib2Me)-Sp-dC^{m}-Sp-dG^{c}-Sp-dT-Sp-dT-Sp-dC-Sp-
dT-Sp-dC-Sp-dT-Sp-(G-Rib2Me)-Sp-(U-Rib2Me)-
3′•17 Na⁺

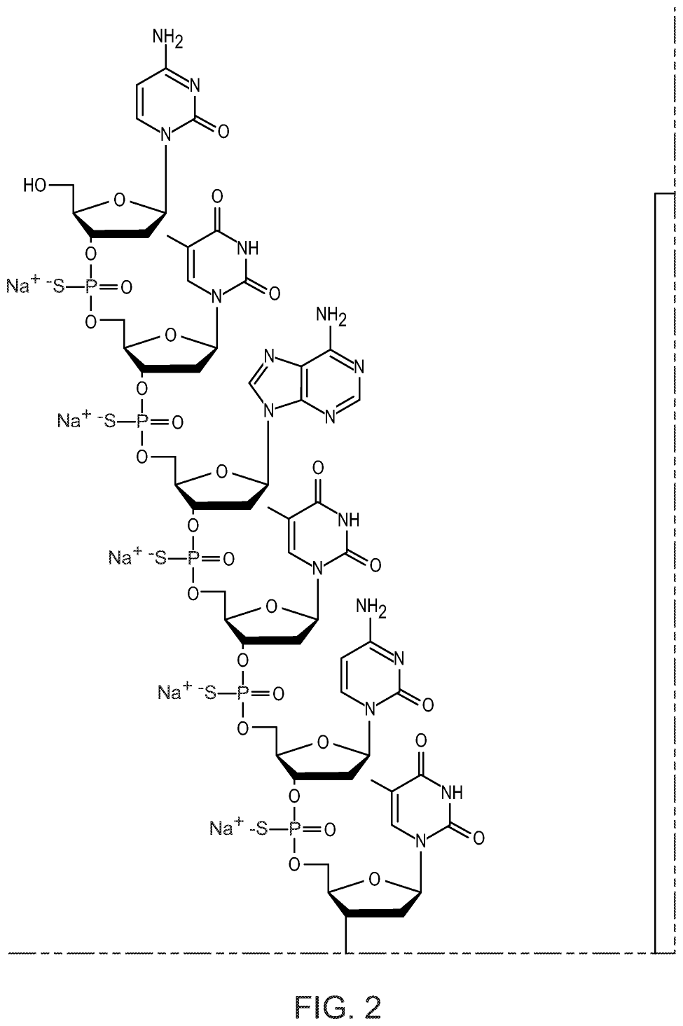

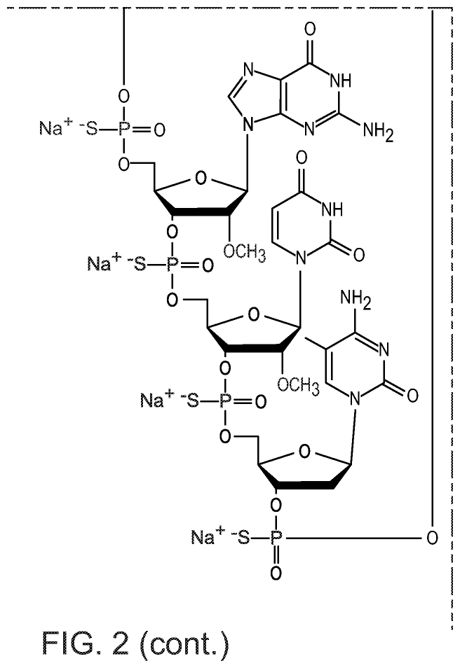

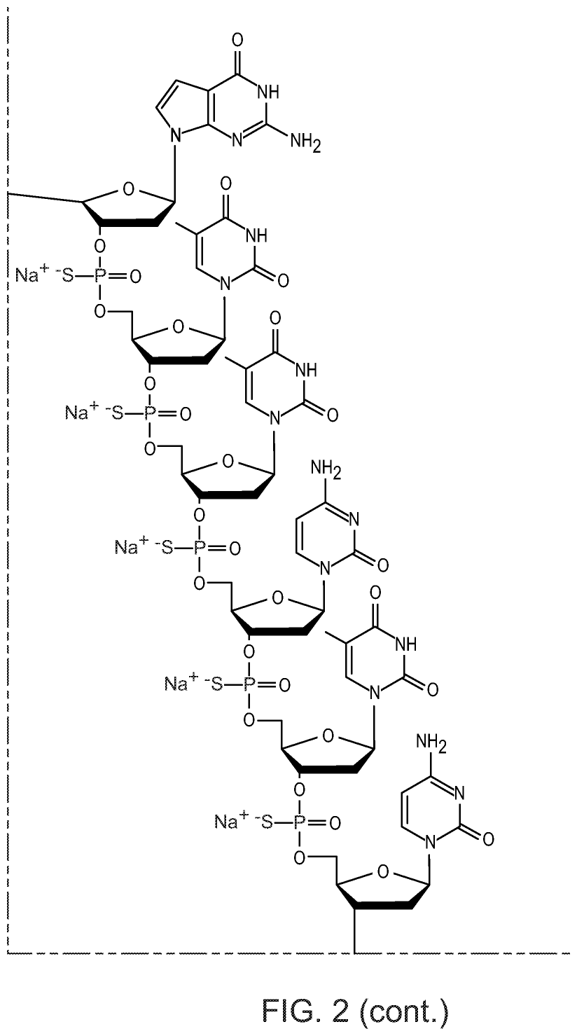

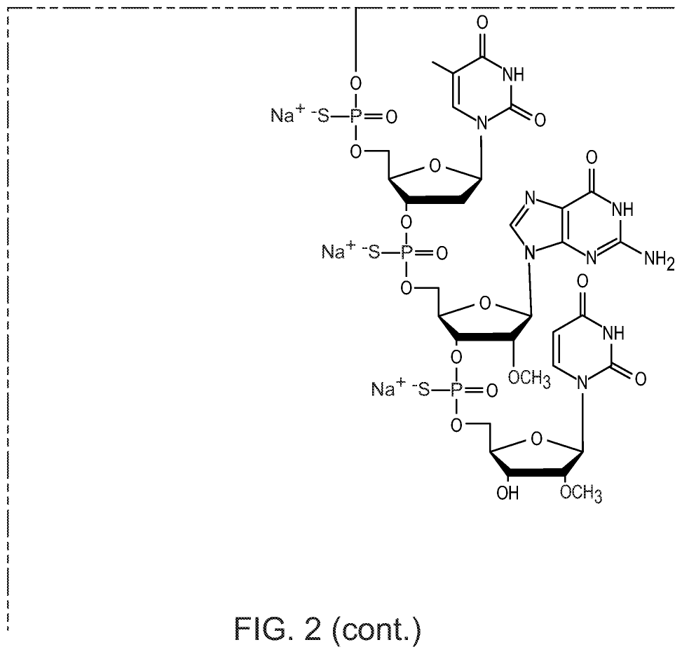

In a tenth example embodiment, the oligonucleotide used in the method described herein in the sodium salt of the oligonucleotide represented by structural formula (III) and is commonly referred to in the art as Bazlitoran or IMO-8400. The molecular weight of the non-salt form of IMO-8400 is 5800.67 g/mol and the molecular formula of non-salt form of IMO-8400 is C₁₇₉H₂₃₃N₅₂O₁₀₁P₁₇S₁₇. A sodium salt of Bazlitoran also referred to herein as AVO010 is represented by the structural formula shown in FIG. 2. The IUPAC name for the compound represented by the structural formula depicted in FIG. 2 is 5′-hydroxy-2′-deoxy-P-thiocytidylyl-(3′→5′)-2′-deoxy-P-thiothymidylyl-(3′→5′)-2′-deoxy-P-thioadenylyl-(3′→5′)-2′-deoxy-P-thiothymidylyl-(3′→5′)-2′-deoxy-P-thiocytidylyl-(3′→5′)-2′-deoxy-P-thiothymidylyl-(3′→5′)-2′-O-methyl-P-thioguanylyl-(3′→5′)-2′-O-methyl-P-thiouridylyl-(3′→5′)-2′-deoxy-P-thio-5-methyl-cytidylyl-(3′→5′)-2′-deoxy-P-thio-7-deaza-guanylyl-(3′→5′)-2′-deoxy-P-thiothymidylyl-(3′→5′)-2′-deoxy-P-thiothymidylyl-(3′-5′)-2′-deoxy-P-thiocytidylyl-(3′→5′)-2′-deoxy-P-thiothymidylyl-(3′→5′)-2′-deoxy-P-thiocytidylyl-(3′→5′)-2′-deoxy-P-thiothymidylyl-(3′→5′)-2′-O-methyl-P-thioguanylyl-(3′→5′)-3′-hydroxy-2′-O-methyluridine 17 sodium salt.

As used herein, the term “ribonucleoside” refers to a compound having the following structural formula:

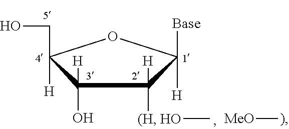

wherein the Base can be any one of nitrogenous bases, such as pyrimidine- or purine-derived bases, and, for example, nucleobases adenine (A), uracil (U), guanine (G), thymine (T), and cytosine (C), each of which can be optionally modified. Unless specifically indicated otherwise, a ribonucleoside includes a 2′-hydroxyl. A 2′-deoxoribonucleoside includes a —CH₂— group at the 2′-position.

### Fatty Liver Disease

Fatty liver disease occurs when there is a buildup of excess fat in the liver. There are two main types of fatty liver disease. The first type of fatty liver disease is non-alcoholic fatty liver disease (NAFLD). The second type of fatty liver disease is alcoholic fatty liver disease (ALD).

NAFLD includes simple fatty liver and non-alcoholic steatohepatitis (NASH). Simple fatty liver means that there is excess fat in the liver, but inflammation is not present. NASH is much more serious than simple fatty liver. NASH means there is fat in the liver and also inflammation and in some instances damage to the liver cells. The inflammation and liver cell damage that happen with NASH cause serious problems such as fibrosis (scarring of the liver), cirrhosis (sever scarring of the liver) and liver cancer.

ALD includes alcoholic hepatitis and alcoholic cirrhosis.

In some embodiments, the method described herein can further comprise improving the NAS score in a subject. The NAS score can be improved by at least 30%.

A “therapeutically effective amount” of an oligonucleotide or salt thereof as described herein is an amount that when administered to a subject with a disease or condition, will have the intended therapeutic effect, e.g., alleviation, amelioration, palliation or elimination of one of more manifestations of the disease or condition in the subject. A therapeutically effective amount of the compound of Formula (I) can be from about 0.5 mg/kg to about 50 mg/kg. For example, from about 0.5 mg/kg to about 25 mg/kg and 0.5 mg/kg to about 20 mg/kg. Suitable doses include about 0.75 mg/kg, about 1.5 mg/kg, about 3 mg/kg and about 6 mg/kg. In a particular embodiment, the dose is 3 mg/kg. In another particular embodiment, the dose is 3 mg/kg and the dose is administered once per week.

As used herein, method of treating means amelioration, prevention or relief from the symptoms and/or effects associated with a disorder or condition. The disorder or condition is fatty liver disease. In one embodiment, the fatty liver disease is non-alcoholic fatty liver disease (NAFLD). In a specific embodiment, the NAFLD is simple fatty liver (steatosis). In another embodiment, the NAFLD is non-alcoholic steatohepatitis (NASH).

### Pharmaceutical Compositions/Methods of Administration

The compositions and methods of the present invention can be utilized to treat a subject in need thereof. In certain embodiments, the subject is a mammal such as a human, or a non-human mammal. When administered to subject, such as a human, the composition or the compound is preferably administered as a pharmaceutical composition comprising, for example, a compound of the invention and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In a specific embodiment, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophilizate for reconstitution, powder, solution, syrup, suppository, injection or the like. The composition can also be present in a transdermal delivery system, e.g., a skin patch. The composition can also be present in a solution suitable for topical administration, such as an eye drop.

A pharmaceutically acceptable carrier can contain physiologically acceptable agents that act, for example, to stabilize, increase solubility or to increase the absorption of a compound such as a compound of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a self-emulsifying drug delivery system or a self-microemulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of a subject without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the subject. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); anally, rectally or vaginally (for example, as a pessary, cream or foam); parenterally (including intramuscularly, intravenously, subcutaneously or intrathecally as, for example, a sterile solution or suspension); nasally; intraperitoneally; subcutaneously; transdermally (for example as a patch applied to the skin); and topically (for example, as a cream, ointment or spray applied to the skin, or as an eye drop). The compound may also be formulated for inhalation. In certain embodiments, a compound may be simply dissolved or suspended in sterile water.

The formulations may conveniently be presented in unit dosage form and may be prepared by any methods well known in the art of pharmacy. The amount of active ingredient which can be combined with a carrier material to produce a single dosage form will vary depending upon the subject being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions for rectal, vaginal, or urethral administration may be presented as a suppository, which may be prepared by mixing one or more active compounds with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the pharmaceutical compositions for administration to the mouth may be presented as a mouthwash, or an oral spray, or an oral ointment.

Alternatively or additionally, compositions can be formulated for delivery via a catheter, stent, wire, or other intraluminal device. Delivery via such devices may be especially useful for delivery to the bladder, urethra, ureter, rectum, or intestine.

Formulations which are suitable for vaginal administration also include pessaries, tampons, creams, gels, pastes, foams or spray formulations containing such carriers as are known in the art to be appropriate.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinacious biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the subject being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the subject's condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods of the invention will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present invention, the active compound may be administered two or three times daily. In particular embodiments, the active compound will be administered once daily.

In certain embodiments, compounds of the invention may be used alone or conjointly administered with another type of therapeutic agent. As used herein, the phrase “conjoint administration” refers to any form of administration of two or more different therapeutic compounds such that the second compound is administered while the previously administered therapeutic compound is still effective in the body (e.g., the two compounds are simultaneously effective in the subject, which may include synergistic effects of the two compounds). For example, the different therapeutic compounds can be administered either in the same formulation or in a separate formulation, either concomitantly or sequentially. In certain embodiments, the different therapeutic compounds can be administered within one hour, 12 hours, 24 hours, 36 hours, 48 hours, 72 hours, or a week of one another. Thus, a subject who receives such treatment can benefit from a combined effect of different therapeutic compounds.

In certain embodiments, conjoint administration of compounds of the invention with one or more additional therapeutic agent(s) provides improved efficacy relative to each individual administration of the compound of the invention or the one or more additional therapeutic agent(s). In certain such embodiments, the conjoint administration provides an additive effect, wherein an additive effect refers to the sum of each of the effects of individual administration of the compound of the invention and the one or more additional therapeutic agent(s).

This invention includes the use of pharmaceutically acceptable salts of compounds of the invention in the compositions and methods of the present invention. A “pharmaceutically acceptable salt” means any non-toxic salt that, upon administration to a recipient, is capable of providing, either directly or indirectly, a compound of this invention. A “pharmaceutically acceptable counterion” is an ionic portion of a salt that is not toxic when released from the salt upon administration to a recipient. In certain embodiments, contemplated salts of the invention include, but are not limited to, Na, Ca, K, Mg, Zn or other metal salts. In a particular embodiment, the salts of the invention (the salts of the compound represented by Formula (I)) is the sodium salt.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

Unless defined otherwise, all technical and scientific terms used herein have the same meaning as commonly understood by one having ordinary skill in the art to which this disclosure belongs. Although other compounds or methods can be used in practice or testing, certain preferred methods are now described in the context of the following preparations and schemes.

### Experimental Methods

### Efficacy of IMO-8400 in Diet Induced NAFLD/NASH Cynomolgus Monkeys

The purpose of this study was to determine the efficacy of IMO-8400 in diet induced NAFLD/NASH male cynomolgus monkeys following subcutaneous (SC) administration of IMO-8400 at 3 mg/kg once weekly for 12 consecutive weeks.

Twelve NAFLD/NASH male cynomolgus monkeys were identified based on liver histophathology and cfDNA levels. The animals were randomly divided into the following groups:
Group 1: n=5; administered vehicle (sterile saline) by subcutaneous injection once weekly for 12 consecutive weeks.
Group 2: n=7; administered IMO-8400 solution by subcutaneous injection once weekly for 12 consecutive weeks.

Results are presented in detail below and include the following observations:
a) Adiponectin Levels: Adiponectin levels in 6 out of 7 animals administered IMO-8400 were noticeably elevated at week 12 as compared to pre-dose (baseline) levels.
b) Body Weight: Compared to day 1, the mean animal body weight was decreased 3.27% in Group 1 (Control) and 10.87% in Group 2 (Treated) on day 84 (end of treatment).
c) NAS Score: 4 of 7 animals in Group 2 (Treated) had a clinically meaningful 2-point or more reduction in their NAS Score. Only 1 of 5 animals in Group 1 (Control) had a 2-point reduction.

Dose Administration: Animals were weighed prior to dose administration on each day of dosing to calculate the actual dose volume. Subcutaneous injections were made on the animal's back using an approximately 26-gauge needle. All animals in Group 1 received subcutaneous administration of sterile saline once weekly for 12 weeks. The animals in Group 2 received subcutaneous administration of IMO-8400 solution once weekly for 12 weeks.

Plasma Preparation: Whole blood (˜10 mL) was collected from overnight fasted animals in the morning on Day 1-predose, Day 42 and Day 84. Samples were collected into commercial vacuum tubes containing EDTA-K2 as anticoagulant. The collected blood samples were placed on wet ice, and processed for plasma by centrifugation at approximately 4° C., 3000 g for 15 minutes within 30 minutes of collection. The plasma supernatant was stored at −60° C. until analysis.

Adiponectin levels were detected by Human HMW Adiponectin/Acrp30 Quantikine ELISA Kit (Lot No. P172678, R&D Systems).

analysis.

NAS scores were evaluated by standard criteria as described in: Brunt E M, Kleiner D E, Wilson L A, Belt P, Neuschwander-Tetri B A; NASH Clinical Research Network (CRN). Nonalcoholic fatty liver disease (NAFLD) activity score and the histopathologic diagnosis in NAFLD: distinct clinicopathologic meanings. Hepatology 2011; 53: 810-820; and Kleiner D E, Brunt E M, Van Natta M, Behling C, Contos M J, Cummings O W, Ferrell L D, Liu Y C, Torbenson M S, Unalp-Arida A, Yeh M, McCullough A J, Sanyal A J; Nonalcoholic Steatohepatitis Clinical Research Network. Design and validation of a histological scoring system for nonalcoholic fatty liver disease. Hepatology 2005; 41:1313-1321.

The chart below provides the framework for scoring.

NAFLD Activity Score (NAS)⁰⁻³

| Histopathology | NAS Scoring Criteria | | | | |
|---|---|---|---|---|---|
| feature | Score | 0 | 1 | 2 | 3 |
| Grade of steatosis | Category | <5% | 5-33% | >33-66% | >66% |
| (Low- to medium- | definition | | | | |
| power evaluation | | | | | |
| of parenchymal | | | | | |
| involvement by | | | | | |
| steatosis) | | | | | |
| | Score | 0 | 1 | 2 | |
| Grade of | Category | None | Few | Many | |
| hepatocyte | definition | | ballooning | hepatocytes/ | |
| (ballooning) | | | hepatocytes | prominent | |
| | | | | ballooning | |
| | Score | 0 | 1 | 2 | 3 |
| Grade of lobular | Category | None | <2 foci per | 2-4 foci per | >4 foci per |
| (Inflammation, | definition | | 200X field | 200X field | 200X field |
| Overall assessment | | | | | |
| of all inflammatory | | | | | |
| foci) | | | | | |

NAS total score: The grade of steatosis (0-3), lobular inflammation (0-3) and ballooning (0-2) were then combined to determine the NAFLD activity score (0-3) as proposed.
Histopathological Analysis: Probable or definite NASH (>5), Uncertain (3-4), Not NASH (<2)

Results showing Adiponectin levels for both the Control Group (Group 1) and the Treated Group (Group 2) are shown in Table 1 below.

TABLE 1

| ng/mL | Baseline | Week 6 | Week 12 | Delta mid | Delta End |
|---|---|---|---|---|---|
| Control-1 | 8200 | 6370 | 9902 | −1831 | 1702 |
| Control-2 | 12928 | 10706 | 13554 | −2222 | 626 |
| Control-3 | 5520 | 5905 | 21442 | 385 | 15922 |
| Control-4 | 33107 | 29111 | 21050 | −3996 | −12057 |
| Control-5 | 23280 | 15411 | 23740 | −7869 | 460 |
| Treat-1 | 4781 | 8173 | 8973 | 3392 | 4192 |
| Treat-2 | 16041 | 19257 | 28451 | 3216 | 12410 |
| Treat-3 | 54142 | 47556 | 54806 | −6586 | 664 |
| Treat-4 | 6219 | 12574 | 24074 | 6355 | 17856 |
| Treat-5 | 4393 | 5127 | 8762 | 734 | 4369 |
| Treat-6 | 7947 | 11662 | 14661 | 3715 | 6714 |
| Treat-7 | 971 | 1255 | 5107 | 283 | 4136 |

### Human Levels: 2-38 μg/ml Depending on Sex and BMI

Results showing NAS scoring for both the Control Group (Group 1) and the Treated Group (Group 2) are shown in TABLE 2 below.

TABLE 2

| | | Base | Line | | End | Line | | Total | | |
|---|---|---|---|---|---|---|---|---|---|---|
| | # | ST | BAL | INF | ST | BAL | INF | PRE | POS | DELT |
| PBO | 1001 | 3 | 0 | 0 | 2 | 0 | 0 | 3 | 2 | 1 |
| | 1002 | 3 | 1 | 1 | 2 | 1 | 0 | 5 | 3 | 2 |
| | 10003 | 3 | 1 | 0 | 2 | 1 | 0 | 4 | 3 | 1 |
| | 10004 | 2 | 0 | 1 | 2 | 0 | 1 | 3 | 3 | 0 |
| | 10005 | 2 | 0 | 0 | 1 | 0 | 0 | 2 | 1 | 1 |
| TLR9 | 2001 | 3 | 1 | 0 | 2 | 1 | 1 | 4 | 4 | 0 |
| | 2002 | 2 | 0 | 0 | 2 | 1 | 0 | 2 | 3 | −1 |
| | 2003 | 3 | 0 | 2 | 3 | 0 | 2 | 5 | 5 | 0 |
| | 2004 | 3 | 1 | 0 | 1 | 0 | 0 | 4 | 1 | 3 |
| | 2005 | 3 | 1 | 0 | 1 | 0 | 1 | 4 | 2 | 2 |
| | 2006 | 3 | 1 | 0 | 1 | 0 | 0 | 4 | 1 | 3 |
| | 2007 | 3 | 1 | 1 | 1 | 0 | 0 | 5 | 1 | 4 |

In Table 2, ST=Steatosis, BAL=Ballooning and INF=Inflammation

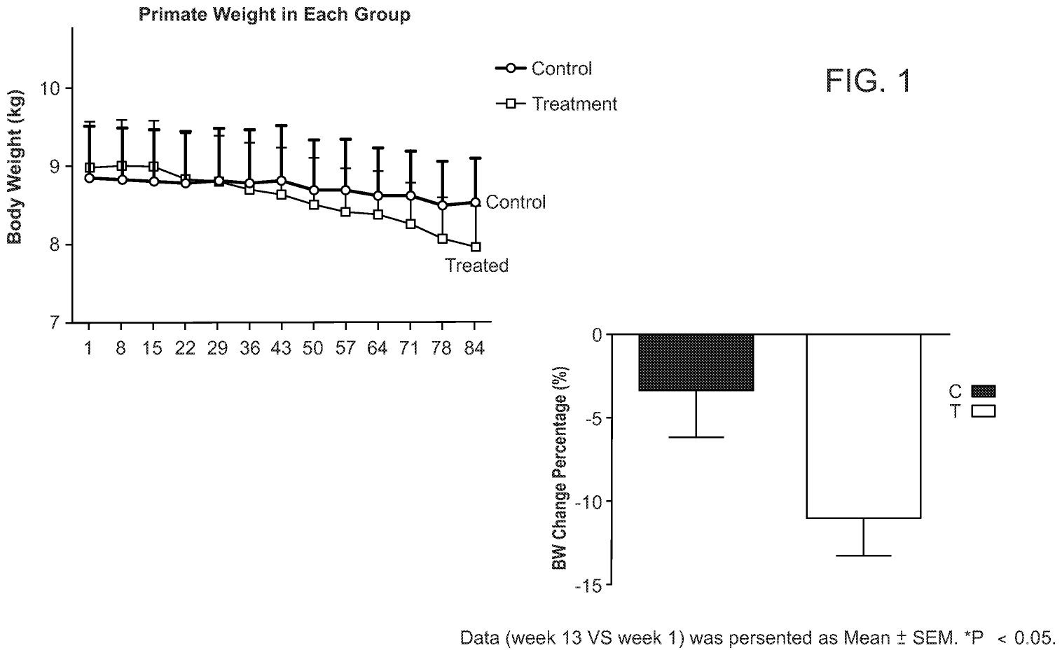

Results of weight loss over the 12 week testing period for both the Control Group (Group 1) and the Treated Group (Group 2) are shown in FIG. 1.

What is claimed is:

1. A method of treating a fatty liver disease in a subject in need thereof comprising administering to the subject a therapeutically effective amount of a compound represented by Formula (I):
5′-N¹N²N³N⁴N⁵N⁶-N⁷N⁸-dC^{m}-dG^{c}-N¹¹N¹²N¹³N¹⁴N¹⁵N¹⁶-N¹⁷N¹⁸-3′ (I)
or a pharmaceutically acceptable salt thereof
wherein, N¹ through N⁶ and N¹¹ through N¹⁶, each independently, is a 2′-deoxoribonucleoside;
N⁷, N⁸, N¹⁷, and N¹⁸, each independently, is a ribonucleoside;
dC^{m} is a 5-methyl-cytosine 2′-deoxoriboside of the following structural formula: 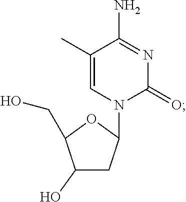 [SMILES: Cc1cn(C2CC(O)C(CO)O2)c(=O)nc1N]
dG^{c} is a 7-deaza-guanine 2′-deoxoriboside of the following structural formula: 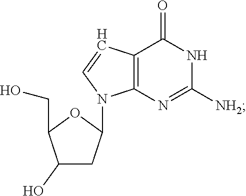 [SMILES: Nc1nc2c(ccn2C2CC(O)C(CO)O2)c(=O)[nH]1]
and
any two adjacent ribonucleosides are covalently linked by either a phosphodiester or a phosphorothioate linker.

2. The method of claim 1, wherein the N⁷ is a guanine 2′-methoxy-riboside represented by the following structural formula:

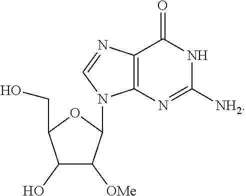 [SMILES: COC1C(O)C(CO)OC1n1cnc2c(=O)[nH]c(N)nc21]

3. The method of claim 1 or claim 2, wherein N⁸ is a uracil 2′-methoxy-riboside represented by the following structural formula:

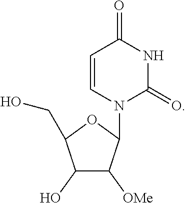 [SMILES: COC1C(O)C(CO)OC1n1ccc(=O)[nH]c1=O]

4. The method of any one of claims 1-3, wherein, N¹⁷ is a guanine 2′-methoxy-riboside.

5. The method of any one of claims 1-4, wherein, N¹⁸ is a uracil 2′-methoxy-riboside.

6. The method of claim 1, wherein N⁷ is a guanine 2′-methoxy-riboside, N⁸ is a uracil 2′-methoxy-riboside, N¹ is a guanine 2′-methoxy-riboside, and N¹⁸ is a uracil 2′-methoxy-riboside.

7. The method of any one of claims 1-6, wherein N¹ is cytosine 2′-deoxoriboside, N² is thymine 2′-deoxoriboside, N³ is adenine 2′-deoxoriboside, N⁴ is thymine 2′-deoxoriboside, N⁵ is cytosine 2′-deoxoriboside, and N⁶ is thymine 2′-deoxoriboside.

8. The method of any one of claims 1-7, wherein N¹¹ is thymine 2′-deoxoriboside, N¹² is thymine 2′-deoxoriboside, N¹³ is cytosine 2′-deoxoriboside, N¹⁴ is thymine 2′-deoxoriboside, N¹⁵ is cytosine 2′-deoxoriboside, and N¹⁶ is thymine 2′-deoxoriboside.

9. The method of claim 1, wherein the oligonucleotide of Formula (I) is represented by the following structural formula: 5′-dC-dT-dA-dT-dC-dT-(G-Rib2Me)-(U-Rib2Me)-dC^{m}-dG^{c}-dT-dT-dC-dT-dC-dT-(G-Rib2Me)-(U-Rib2Me)-3′ (II) or a pharmaceutically acceptable salt thereof.

10. The method of claim 1, wherein the oligonucleotide of Formula (I) is represented by the following structural formula: 5′-dC-Sp-dT-Sp-dA-Sp-dT-Sp-dC-Sp-dT-Sp-(G-Rib2Me)-Sp-(U-Rib2Me)-Sp-dC^{m}-Sp-dG^{c}-Sp-dT-Sp-dT-Sp-dC-Sp-dT-Sp-dC-Sp-dT-Sp-(G-Rib2Me)-Sp-(U-Rib2Me)-3′ (III) or a pharmaceutically acceptable salt thereof, wherein Sp represents a phosphorothioate linker in the “Sp” configuration.

11. The method of any one of claims 1-10, wherein the oligonucleotide is in the form of the sodium salt.

12. The method of claim 1, wherein the oligonucleotide is Bazlitoran.

13. The method of any one of claims 1-12, wherein the fatty liver disease is non-alcoholic fatty liver disease.

14. The method of claim 13, wherein the non-alcoholic fatty liver disease is non-alcoholic steatohepatitis.

15. A method of identifying the likelihood of a fatty liver disease in a subject to be responsive to administration of a compound of Formula (I):
(I)
5′-N¹N²N³N⁴N⁵N⁶-N⁷N⁸-dC^{m}-dG^{c}-N¹¹N¹²N¹³N¹⁴N¹⁵N¹⁶-N¹⁷N¹⁸-3′
or a pharmaceutically acceptable salt thereof wherein, N¹ through N⁶ and N¹¹ through N¹⁶, each independently, is a 2′-deoxoribonucleoside;
N⁷, N⁸, N¹⁷, and N¹⁸, each independently, is a ribonucleoside;
dC^{m} is a 5-methyl-cytosine 2′-deoxoriboside of the following structural formula: 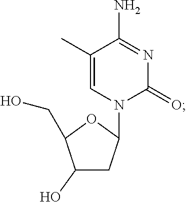 [SMILES: Cc1cn(C2CC(O)C(CO)O2)c(=O)nc1N]
dG^{c} is a 7-deaza-guanine 2′-deoxoriboside of the following structural formula: 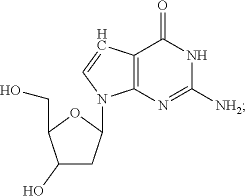 [SMILES: Nc1nc2c(ccn2C2CC(O)C(CO)O2)c(=O)[nH]1]
and
any two adjacent ribonucleosides are covalently linked by either a phosphodiester or a phosphorothioate linker, the method comprising:
a) obtaining or providing a plasma sample from a subject having a fatty liver disease;
b) measuring the plasma adiponectin level in the sample; and
c) comparing the plasma adiponectin level to a control level of adiponectin;
wherein if the plasma adiponectin level is less than the control level the fatty liver disease is identified as being more likely to be responsive to the administration of the compound of Formula (I).

16. The method of claim 15, wherein the control level of adiponectin is 38 μg/mL.

17. A method of assessing the efficacy of treating fatty liver disease with a compound of Formula (I) in a subject in need thereof:
(I)
5′-N¹N²N³N⁴N⁵N⁶-N⁷N⁸-dC^{m}-dG^{c}-N¹¹N¹²N¹³N¹⁴N¹⁵N¹⁶-N¹⁷N¹⁸-3′
or a pharmaceutically acceptable salt thereof wherein,
N¹ through N⁶ and N¹¹ through N¹⁶, each independently, is a 2′-deoxoribonucleoside;
N⁷, N⁸, N¹⁷, and N¹⁸, each independently, is a ribonucleoside;
dC^{m} is a 5-methyl-cytosine 2′-deoxoriboside of the following structural formula: 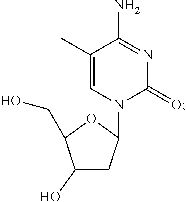 [SMILES: Cc1cn(C2CC(O)C(CO)O2)c(=O)nc1N]
dG^{c} is a 7-deaza-guanine 2′-deoxoriboside of the following structural formula: 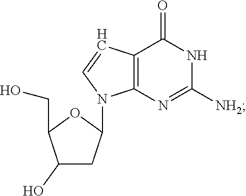 [SMILES: Nc1nc2c(ccn2C2CC(O)C(CO)O2)c(=O)[nH]1]
and
any two adjacent ribonucleosides are covalently linked by either a phosphodiester or a phosphorothioate linker, for treating fatty liver disease the method comprising:
a) detecting in a subject plasma sample at a first point in time the level of adiponectin;
b) repeating step a) during at least one subsequent point in time after administration of the compound represented by Formula (I); and
c) comparing the levels detected in steps a) and b), wherein an increased level of adiponectin relative to at least one subsequent subject plasma sample, indicates that the compound of Formula (I) treats the fatty liver disease in the subject.
